(19) United States
(12) Patent Application Publication
(10) Pub. No.: US 2011/0033848 A1
(43) Pub. Date: Feb. 10, 2011

(54) EPIGENETIC METHODS

(75) Inventor: Malcolm James Simons, Victoria (AU)

Correspondence Address: FOLEY AND LARDNER LLP;SUITE 500, 3000 K STREET NW, WASHINGTON, DC, 20007, US

(73) Assignee: Simons Haplomics Limited

(21) Appl. No.: 12/663,197

(22) PCT Filed: Jun. 6, 2008
(86) PCT No.: PCT/AU08/00806

§ 371 Date: Feb. 5, 2010

(30) Foreign Application Priority Data
Jun. 7, 2007 (AU) 2007903075

(51) Int. Cl. C12Q 1/68; G01N 33/53

(52) U.S. Cl. 435/6; 435/7.21

(57) ABSTRACT

The present invention provides methods for obtaining epigenetic information for a polyploid subject, the method including the steps of obtaining a biological sample from the subject, the sample containing: (i) at least one paternally-derived DNA molecule and/or associated protein and/or, (ii) at least one maternally-derived DNA molecule and/or associated protein, analyzing any one or more of the paternally- or maternally-derived DNA molecules or associated proteins for the presence or absence of modifications, wherein the step of analyzing determines whether any two modifications are present in cis on one chromosome, or in trans across two sister chromosomes.

### FIELD OF THE INVENTION

The present invention relates to the field of genetics, and more specifically epigenetics. In particular, the invention relates to methods for investigating epigenetic characteristics of the haploid state of an organism.

### BACKGROUND TO THE INVENTION

Epigenetic inheritance is the transmission of information from a cell or multicellular organism to descendants without that information being encoded in the nucleotide sequence of the gene. One type of epigenetic inheritance is DNA methylation, which has been demonstrated to be involved in a number of human diseases. For example, changes in DNA methylation profiles are common in disorders such as cancer, Beckwith-Wiedemann, Prader-Willi and Angelman syndromes. DNA methylation is also known to be involved in modulating gene expression in the course of human development. It is thought that heavy methylation of promoter regions is important in down regulation of transcription, thereby providing a “switch” for gene expression.

DNA methylation is an epigenetic modification that typically occurs at CpG sites (that is, where a cytosine is directly followed by a guanine in the DNA sequence); the methylation results in the conversion of the cytosine to 5-methylcytosine. The formation of Me-CpG is catalyzed by the enzyme DNA methyltransferase. CpG sites are uncommon in vertebrate genomes but are often found at higher density near vertebrate gene promoters where they are collectively referred to as CpG islands. The methylation state of these CpG sites can have a major impact on gene activity and/or expression.

The pattern of methylation has recently become an important topic for research. For instance, both DNA hypomethylation (a loss of methylation) and DNA hypermethylation (an increase in methylation) have been linked to studies examining genes that are differentially methylated between normal and cancerous tissue. The cancer-related genes that have been linked to altered methylation include those involved in cell cycle regulation, DNA repair, RAS signaling and invasion. Studies have found that in normal tissue, methylation of a gene is mainly localised to the coding region, which is CpG poor. In contrast, the promoter region of the gene is unmethylated, despite a high density of CpG islands in the region.

The degree of methylation may also be important in gene regulation. In cancer, epigenetically mediated gene silencing occurs gradually. It begins with a subtle decrease in transcription, fostering a decrease in protection of the CpG island from the spread of flanking heterochromatin and methylation into the island. This loss results in gradual increases of individual CpG sites, which vary between copies of the same gene in different cells.

Another type of epigenetic inheritance is that arising from the effect of DNA-associated proteins. It is known, for example, that the acetylation status of histones can affect expression of the gene with which they are associated. It is thought that acetylation of certain histones leads to silencing of the gene due to the more dense packaging of DNA in the chromatin structure.

While the prior art has disclosed a link between epigenetic modifications to DNA and associated proteins, and phenotype, the interactions are complex and as yet not fully elucidated. It is an aspect of the present invention to overcome or alleviate a problem of the prior art to provide methods for utilizing patterns of epigenetic modifications to DNA and associated proteins patterns to ascribe phenotypes to organisms than that previously thought possible.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect the present invention provides a method for obtaining epigenetic information for a polyploid subject, the method including the steps of obtaining a biological sample from the subject, the sample containing: (i) at least one paternally-derived DNA molecule and/or associated protein and/or, (ii) at least one maternally-derived DNA molecule and/or associated protein, analyzing any one or more of the paternally- or maternally-derived DNA molecules or associated proteins for the presence or absence of modifications, wherein the step of analyzing determines whether any two modifications are present in cis on one chromosome, or in trans across two sister chromosomes.

Applicant proposes that epigenetic analysis considers maternally-derived DNA (and associated proteins) and paternally-derived DNA (and associated proteins) separately. In this way, it is possible to determine a definitive epigenetic characterization of the subject, with this characterisation providing new links between disease and the epigenetic state of a subject, for example. By contrast, methods of the prior art provide an “averaged” result since the epigenetic modifications of the maternal and paternal DNA and associated proteins is summed.

In one form of the method the step of analyzing determines whether the modifications can be ascribed to the paternally-derived DNA and/or associated protein, or the maternally-derived DNA and/or associated protein. In another embodiment, the presence or absence of the modifications is capable of modulating expression of the DNA molecule in vivo.

In one form of the method the step of analyzing includes the substantial isolation of a paternally-derived DNA and/or associated protein from a maternally-derived DNA and/or associated protein using a physical method. The physical method may be a laser-mediated dissection of the paternally-derived DNA molecule and/or associated protein from the maternally-derived DNA molecule and/or associated protein.

The step of analyzing may also include an in situ method capable of selectively analyzing paternally-derived DNA and/or associated protein as compared with maternally-derived DNA and/or associated protein. The in situ method allows for probing of polyploid material to provide definitive haploid epigenetic information, making it unnecessary to physically separate the maternal and paternal DNA molecules.

In another form of the method the DNA molecule or associated protein is present in, or obtained, from a diploid cell. While gametes contain haploid information, these cells can be difficult to obtain in the clinic and/or provide incorrect information on epigenetic modifications present in somatic cells.

In another form of the invention where the step of analyzing is performed on DNA, the modification is methylation. The analysis may be implemented using any suitable methodology, however typically the step of analyzing the one or more sites for the presence or absence of methylation comprises a method selected from the group consisting of DNA sequencing using bisulfite treatment, restriction landmark genomic scanning, methylation-sensitive arbitrarily primed PCR, Southern analysis using a methylation-sensitive restriction enzyme, methylation-specific PCR, restriction enzyme digestion of PCR products amplified from bisulfite-converted DNA, and combinations thereof. Where the analyzing is performed on protein, the protein may be a histone and the modification may be acetylation.

The epigenetic information provided by the present method may be capable of providing phenotypic information for the subject such as the presence or absence of a disease, condition, or disorder; a predisposition to a disease, condition, or disorder; the ability or inability to respond to a potentially therapeutic molecule; the ability or inability to mount an immune response against a foreign antigen or a self-antigen; the presence or absence of an allergy; or a predisposition to an allergy.

### DETAILED DESCRIPTION OF THE INVENTION

In a first aspect the present invention provides a method for obtaining epigenetic information for a polyploid subject, the method including the steps of obtaining a biological sample from the subject, the sample containing: (i) at least one paternally-derived DNA molecule and/or associated protein and/or, (ii) at least one maternally-derived DNA molecule and/or associated protein, analyzing any one or more of the paternally- or maternally-derived DNA molecules or associated proteins for the presence or absence of modifications, wherein the step of analyzing determines whether any two modifications are present in cis on one chromosome, or in trans across two sister chromosomes.

To the best of the Applicant's knowledge, the invention disclosed herein is the first time that the importance of phase when studying epigenetic inheritance by way of DNA and protein modifications has been appreciated. For the first time, phase-specific information is obtained on epigenetic phenomena such as methylation of DNA and acetylation of DNA associated proteins. The present invention therefore provides the ability to discern whether an epigenetic modification at two sites are present on the same chromosome (i.e. a cis relationship), or alternatively one site is present on the paternally-derived chromosome and the other on the maternally-derived sister chromosome (i.e. a trans relationship). Applicant proposes that the presently accepted methods of epigenetic analysis using both maternally-derived DNA and paternally-derived DNA results in a loss of information by providing an “averaged” result. This information is important in inter alia identifying phase-specific epigenetic effects in individuals and populations.

The field of epigenetics is relatively new in the art of genetics, and refers to the study of changes in genome function that do not rely on the specific nucleotide sequence within the DNA of an organism. Epigenetics includes the study of effects that are inherited from one cell generation to the next whether these occur in embryonic morphogenesis, regeneration, normal turnover of cells, tumors, cell culture, or the replication of single celled organisms. Specific epigenetic processes of interest include paramutation, bookmarking, gene silencing, X chromosome inactivation, position effect, reprogramming, transvection, imprinting, maternal effects, the progress of carcinogenesis, the effects of many teratogens, and the regulation of histone modifications and heterochromatin.

In one form of the invention the step of analyzing determines whether the modifications can be ascribed to the paternally-derived DNA and/or associated protein, or the maternally-derived DNA and/or associated protein. Such a determination may not be strictly necessary given that the only information required may be whether any two modifications are present in cis or trans.

In another form of the method the presence or absence of the modifications is capable of modulating expression of the DNA molecule in vivo. As appreciated by the skilled person, the expression of genes is controlled at least in part by epigenetic modifications such as DNA methylation. DNA methylation is one epigenetic modification of DNA that is proposed to be universal in eukaryotes. In humans, approximately 1% of DNA bases undergo DNA methylation. In adult somatic tissues, DNA methylation typically occurs in a CpG dinucleotide context; non-CpG methylation is prevalent in embryonic stem cells. In plants, cytosines are methylated both symmetrically (CpG or CpNpG) and asymmetrically (CpNpNp), where N can be any nucleotide.

In mammals, between 60-70% of all CpGs are methylated. Unmethylated CpGs are grouped in clusters called “CpG islands” that are present in the 5′ regulatory regions of many genes. In many disease processes such as cancer, gene promoter CpG islands acquire abnormal hypermethylation, which results in heritable transcriptional silencing. Reinforcement of the transcriptionally silent state is mediated by proteins that can bind methylated CpGs. These proteins, which are called methyl-CpG binding proteins, recruit histone deacetylases and other chromatin remodelling proteins that can modify histones, thereby forming compact, inactive chromatin termed heterochromatin. This link between DNA methylation and protein via alteration to chromatin structure is related to the development of various phenotypes. For example, loss of Methyl-CpG-binding Protein 2 (MeCP2) has been implicated in Rett syndrome and Methyl-CpG binding domain protein 2 (MBD2) mediates the transcriptional silencing of hypermethylated genes in cancer. While there may be an interaction between DNA-associated proteins and methylation, it will be understood that the present invention includes the analysis of DNA-associated proteins that have no relationship to DNA methylation.

As described above, proteins associated with DNA may be involved in the modulation of gene expression. For example, the physical structure of the DNA, as it exists compacted into chromatin, can affect the ability of transcriptional regulatory proteins (termed transcription factors) and RNA polymerases to find access to specific genes and to activate transcription from them.

Chromatin is a term designating the structure in which DNA exists within cells. The structure of chromatin is determined and stabilized through the interaction of the DNA with DNA-binding proteins. There are 2 classes of DNA-binding proteins. The histones are the major class of DNA-binding proteins involved in maintaining the compacted structure of chromatin. There are 5 different histone proteins identified as H1, H2A, H2B, H3 and H4.

The other class of DNA-binding proteins is a diverse group of proteins simply referred to as non-histone proteins. This class of proteins includes the various transcription factors, polymerases, hormone receptors and other nuclear enzymes. In any given cell there are greater than 1000 different types of non-histone proteins bound to the DNA.

The binding of DNA by the histones generates a structure called the nucleosome. The nucleosome core contains an octamer protein structure consisting of 2 subunits each of H2A, H2B, H3 and H4. Histone H1 occupies the internucleosomal DNA and is identified as the linker histone. The nucleosome core contains approximately 150 by of DNA. The linker DNA between each nucleosome can vary from 20 to more than 200 bp. These nucleosomal core structures would appear as beads on a string if the DNA were pulled into a linear structure.

The nucleosome cores themselves coil into a solenoid shape which itself coils to further compact the DNA. These final coils are compacted further into the characteristic chromatin seen in a karyotyping spread. The protein-DNA structure of chromatin is stabilized by attachment to a non-histone protein scaffold called the nuclear matrix.

The present invention includes modifications to any protein that is associated with DNA, and wherein the modification is capable of modulating the expression of the gene with which it is associated. For example, histone post-translational modifications (PTMs) associate with positive and negative transcriptional states. A typical model for the role of these PTMs is that in response to cytoplasmic signalling to transcription factors, positive-acting PTMs are established across promoters and open reading frames by DNA-bound activators and RNA polymerase. Negative-acting marks are made across genes during repression by DNA-bound repressor recruitment across heterochromatic regions of the genome. Both sets of modifications alter the nucleosome surfaces which then recruit regulatory protein complexes.

In the context of the present invention, histone PTMs include, but are not limited to acetylation of histone 3 (H3), histone 4 (H4), histone 2A (H2A), histone 2B (H2B); phosphorylation of H3, H2A and H2B; arginine methylation of H3 and H4; lysine methylation of H3 and H4; lysine ubiquitylation of H2A and H2B; lysine Sumoylation of H2A and H2B; and proline isomerisation in H3; ADP-ribosylation deimination (conversion of arginine to citrulline). The variant histones H2AX, H3.1, H3.3 and Hzt1 are also modified by PTMs.

This repertoire of histone PTMs, known as the “histone code”, serve as binding surfaces for the association of effector proteins containing specific interacting domains. For instance, acteylation is recognised by bromodomains, methylation is recognised by chromo-like domains of the Royal family (chromo, tudor, MBT) and non-related PHD domains, and phosphorylation is recognised by a domain within 14-3-3 proteins.

The histone acetyltransferases (HATs) of interest include the GNAT [GCN5 (general control of amino-acid synthesis 5)-related acetyltransferase] family, the CBP/P300 (CREB-binding protein) family, and the MYST (MOZ, YBF2/SAS3, SAS2, TIP60 protein) family. These HATs, and the transcription factor and nuclear-hormone related histone acetyltransferases, also include GCN5, PCAF (P300/CREB-binding protein-associated factor), GCN5L (general control of amino-acid synthesis 5-like 2), ELP3, P300 (e1a-binding protein p300), CBP (CREB-binding protein), TIP60, MOF/MYST1, MOZ (monocytic leukaemia zinc finger protein)/MYST3, MORF (MOZ-related factor)/MYST4, HB01 (histone acetyltransferase binding to ORC)/MYST2, ATF2, TAF1 (TATA box-associated factor 1), GTF3C4: general transcription factor 3c, polypeptide 4), ACTR: activin receptor, SRC-1 (steroid receptor coactivator 1)/NCOA1/2 (nuclear receptor coactivator 1), ACTR (activin receptor), SRC-1 (steroid receptor coactivator 1), CDYL and HAT1 (histone aceayl transferase 1).

The histone deacetylases (HDACs) of interest include class I and class II HDACs (such as HDACs 1 through 8 and HDAC10), and the class III NAD-dependent enzymes of the Sir family (Such as SirT2).

The histone methyl transferases (HMTs) of interest include both type I and type II arginine HMTs (such as PRMT1, PRMT4, PRMT5 and PRMT7) and lysine HMTs. The lysine HMTs of interest include MLL (Mixed Lineage Leukaemia)-1 through 5, the Set 1 family which have homology to the yeast Set1 protein (including SET1A and SET1B), SET2, SYMD2, Pr-SET 7/8, CLL8, NSD1, DOT1, SUV42H1/2, EZH2, RIZ1, SUV39H1/2, EuHMTase, GLP, ESET, SETDB1, and G9a.

The arginine and lysine demethylases of interest include protein arginine methylatransferases (PMRT4, PRMT5, and CARM1), LSD1, JHDM1a, JHDM1b, JHDM2a, JHDM2b, JMJD2A/JHDM3A, JMJD2B, JMJD2C, and JMJD2D.

Enzymes mediating other histone PTMs such as deimination, phosphorylation, sumoylation and ubiquitylation that are of interest are PADI4, Bmi/Ring1A, RNF20/RNF40 and UbcH6), mono-ADP-ribosyltransferases and poly-ADP-ribose polymerases, proline isomerases and kinases such as haspin, MSK1/2, CKII and Mst1. Of further interest are proteins binding methylated DNA, such as the methyl-CpG binding domain (MBD) proteins, including MeCP2, MBD1-4, and MBD3L-1, and MBD3L-2.

In addition, since RNAi is involved in heterochromatin formation in insects, plants and fungi, and recent work suggests these mechanisms are present in vertebrates, the present invention includes RNAi-mediated chromatin modifications.

The skilled person will be familiar with methods for isolating DNA associated proteins. Overall DNA 5-methylcytosine content or histone PTMs may be examined using high-performance capillary electrophoresis, high performance liquid chromatography or mass spectrometry. Epigenetic analyses such as restriction landmark genomic scanning may be used to examine haploid genetic material, however approaches utilising amplification (usually by polymerase chain reaction) of target sequences, such as amplification of intermethylated sites (AIMS) or differential methylation hybridization (DMH) are of particular interest. Additionally those that utilise amplification of a signal for detection, such as the detection events of P-LISA (discussed below) facilitating rolling-circle amplification of a hybridisation target.

In order to miniaturise methylation analysis of single chromosomes, it is possible that techniques such as proximity-ligation in situ assay (P-LISA) which are capable of detecting zeptomole amounts (40×10⁻²¹ mol) of protein (Fredriksson S et al. (2002) Protein detection using proximity-dependent DNA ligation assays. Nat Biotechnol. 20(5):473-7), may be used. This approach utilises antibodies to the proteins of interest (in one example antibodies against methylated proteins) that, when brought into proximity by binding their target proteins, allow hybridisation of conjugated oligonucleotides which serve as a template for rolling circle amplification upon enzymatic ligation. Since two independent recognition events are required for detection of target proteins, detection is highly specific and capable of detecting individual protein complexes (Soderberg O (2006) Direct observation of individual endogenous protein complexes in situ by proximity ligation. Nat Methods. 2006 3(12):995-1000). This approach has recently been adapted to study protein-DNA interactions, and has been adapted to allow in situ analysis of DNA-protein interactions for localized detection (Gustafsdottir S M et al. (2007) In vitro analysis of DNA-protein interactions by proximity ligation. Proc Natl Acad Sci USA. 104(9):3067-72).

Chromatin immunoprecipitation coupled to gene array technology (ChIP on chip). In this approach, an antibody to the post-translationally modified DNA-binding protein of interest is used to immunoprecipitate the both the modified protein and its DNA target. In brief, crosslinking is used to fix DNA-binding proteins to DNA, and following fragmentation of DNA, immunoprecipitation is used to purify the protein-DNA fragments with specificity determined by the antibody used. Following hydrolysis to reverse the cross-linking, amplification and labeling of DNA is performed, and the DNA is subsequently hybridized to a microarray and analysed to identify the regions of bound by the DNA-binding protein. Alternately, genes of interest may be examined by PCR using gene specific oligonucleotides (known as single-gene ChIP).

Differential methylation hybridization may also be used to identify methylated DNA. In this approach, specialized DNA microarrays comprising cloned CpG islands are utilised. In the case of the present invention, the DNA samples to be compared—two haploid DNA samples (chromosomes, chromatids etc.)—are each digested with a methylation-sensitive restriction enzyme, and polymerase chain reaction amplicons derived from each sample are then hybridized to the CpG island array. Array elements with a stronger hybridization signal in either sample represent differentially-hypermethylated CpG islands, protected from methylation-sensitive restriction enzyme cleavage, and therefore amplified by PCR in the sample.

Further advances in epigenetic analysis allow examination of epigenetic marks, such as DNA methylation, using diminishing amounts of DNA. For the analysis of DNA methylation, methylated-CpG island recovery assay (MIRA), based on the high affinity of the MBD2/MBD3L1 complex for methylated DNA, has been used to purify methylated DNA. In brief, sonicated DNA is incubated with a matrix containing glutathione-S-transferase (GST)-MBD2b in the presence of MBD3L-1, a binding partner of MBD2 that increases the affinity of MBD2 for methylated DNA. Specifically bound DNA is eluted from the matrix and gene-specific PCR reactions are performed to detect CpG island methylation. Alternately, the bound DNA can be examined using microarray analysis. This technique can already detect methylation using 1 ng of DNA. GST fusions of other proteins involved in epigenesis may also be utilised by such ‘pull-down’ approaches, or fusions involving biotin to increase affinity for pull-down matricies. Advances in quantitative methylation analysis such as MethyLight and Pryosequencing, when coupled with bisulphite sequencing, allow detection of ever smaller amounts of methylated DNA, with one approach, HeavyMethyl, allowing detection of 30 pg of methylated DNA.

Furthermore, recent advances in top-down proteomics (the analysis of intact proteins rather than first digesting them to peptides) may allow examination the intact modification patterns of different histones in a given nucleosome.

While there have been many studies on the mechanisms of epigenesis, the prior art has not adequately appreciated the confounding effects of concurrently considering he epigenetic modification of a maternally-derived DNA molecule or protein at the same time as a paternally-derived DNA molecule or protein. This has occurred because the methods of the prior art analyze epigenetic modifications using diploid material.

Applicant proposes herein that a definitive characterization of the epigenetic state of a subject can only be provided by separately analyzing epigenetic modification of DNA and associated proteins in the haploid state. For example, where methylation is the epigenetic modification, use of haploid material resolves phase-specific effects, for recognition of heterozygous phenomena and provides for analysis of quantitative variation over the multiple CpG sites comprising a (unitarily functional) ‘island’, even for variation at a single CpG site. This definitive characterization leads to more accurate phenotypic information, such as the presence and/or predisposition of an individual to certain diseases or the ability of an individual to respond to certain therapeutic molecules.

Considering a more specific example, DNA methylation is an epigenetic feature that is thought to be involved in the pathogenesis of cancer. The following table describes cancer-related genes thought to be methylation sensitive.

| Gene | Map | Methylated | Notes |
|---|---|---|---|
| 14-3-3 Sigma | 1p | Breast and gastric cancers | |
| ABL1 (P1) | 9q34.1 | 50-100% CML, Some ALL | Only methylated when part of the |
| | | | bcr-abl translocation. |
| ABO | 9q34 | cell lines | |
| APC | 5q21 | Colon, gastric and esophageal | One promoter only. Correlation |
| | | cancer | with expression not established. |
| | | | Type A |
| AR | Xq11-12 | Prostate Cancer Cell Lines, Colon | |
| (Androgen Receptor) | | ACFs | |
| BLT1 (Leukotriene | | Various cell lines | |
| B4 Receptor) | | | |
| BRCA1 | 17q21 | 10-20% Breast cancer, some | Cause of transcriptional silencing |
| | | ovarian | in these cells |
| CALCA | 11p15 | 25-75% Colon, lung, hematopoic | One of the first promoter-CpG |
| (Calcitonin) | | neoplasms. | islands demonstarted to be |
| | | | hypermethylated in cancer. |
| CASP8 | 2q33-34 | Neuroblastoma | Corralates with MycN |
| (CASPASE 8) | | | amplification |
| Caveolin 1 | 7q31.1 | Breast cancer cell lines | |
| CD44 | 11pter-p13 | Prostate cancer | |
| CFTR | 7q31.2 | Cell Lines | No primary tumors reported |
| COX2 | 1q25.2-q25.3 | Colon, Breast and prostate cell | Correlates with expression when |
| | | lines. 15% of primary colon | completely methylated. |
| | | cancers | |
| CSPG2 | 5q12-14 | AGING Colon. 70% colon | Secreted proteoglycan, regulated |
| (Versican) | | | by Rb. |
| CX26 | 13q11-q12 | Breast cancer cell lines | |
| (Connexin 26) | | | |
| Cyclin A1 | 13q12.3-q13 | Various cell lines | |
| DBCCR1 | 9q32-33 | 50% Bladder cancer | Slight methylation in normal |
| | | | bladder |
| ECAD | 16q22.1 | 20-70% Breast, Gastric, Thyroid, | Methylation is often |
| (E-cadherin) | | SCC, Leukemias and Liver ca. | heterogeneous and not always |
| | | | correlated with silencing. Also |
| | | | present in some normal stomach |
| | | | and liver samples |
| Endothelin | 13q22 | 60-70% prostate cancer | |
| Receptor B | | | |
| EPHA3 | 3p11.2 | Leukemias | |
| EPO | 7q21 | HeLa Cells | Normal and primary tumors |
| (Erythropoietin) | | | |
| ER | 6q25.1 | AGING colon, liver, heart muscle, | Upstream promoter not in CpG |
| (Estrogen Receptor) | | AoSMC (cultured), brain, AoEC. | island. Nevertheless, there is a |
| | | 100% Colon cancer 20-30% ER- | good correlation with loss of |
| | | breast cancer 60-70% AML/ALL | expression. |
| | | 20-50% CML-BC 20% Lung | |
| | | (NSCLC) 60% GBM | |
| FHIT | 3p14.2 | 10-20% Esophageal SCC | |
| GPC3 (Glypican 3) | Xq26 | Mesothelioma and Ovarian cancer | |
| | | cell lines | |
| GST-pi | 11q13 | 80-100% Prostate, Liver. 30-60% | DNA repair/detoxification |
| | | Colon, Breast, Kidney. | enzyme. |
| H19 | 11p15.5 | 20-50% Wilm's tumors | Imprinted gene. |
| | | | Hypermethylation is associated |
| | | | with apparent loss of imprinting |
| | | | of the IGF2 gene in Wilm's |
| | | | tumor, but not others. |
| H-Cadherin (CDH13) | 16q24.1-24.2 | 45% Lung Cancer, some ovarian | |
| | | cancer | |
| HIC1 | 17p13.3 | Prostate, Breast and Brain,. 80-100% | Candidate tumor-suppressor |
| | | Colon cancer, Prostate, | gene. First gene cloned based |
| | | Breast, GBM. 20-50% Lung, | on finding a CpG island |
| | | Kidney, Liquid tumors. | hypermethylated in cancer. |
| hMLH1 | 2p22 | 10-20% colon, endometrial and | Almost always associated with |
| | | gastric cancers. 0% lung, breast, | microsatellite instability and, in |
| | | GBM, liquid tumors etc. | celllines, mismatch repair |
| | | | deficiency. |
| HOXA5 | 7p15-p14.2 | Breast cancer | |
| IGF2 | 11p15.5 | AGING colon 100% Colon cancer | IGF2 has a large CpG island that |
| (Insulin-Like Growth | | 50% AML | contains the imprinted P2-4 |
| Factor II) | | | promoters |
| IGFBP7 | 4q12 | Murine SV40 T/t antigen-induced | Normal and primary tumors |
| | | hepatocarcinogenesis | |
| IRF7 | 11 | Various cell lines | |
| LKB1 | 19p13.3 | A few colon, testicular and breast | |
| | | (medullary) primary tumors | |
| LRP-2 (Megalin) | 2q24-31 | Various cell lines | |
| MDGI | 1p35-33 | 50-70% Breast cancers | |
| (Mammary-derived | | | |
| growth inhibitor) | | | |
| MDR1 | 7q21.1 | Drug sensitive leukemia cell lines. | Primary tumors |
| MDR3 (PGY3) | 7q21.1 | Various cell lines | |
| MGMT | 10q26 | 25-50% Brain, colon, lung, breast, | Associated with the MER- |
| (O6 methyl guanine | | NHL etc. | phenotype |
| methyl transferase) | | | |
| MT1a | 16q13 | Rat hepatoma | Normal and primary tumors |
| (metallothionein 1) | | | |
| MUC2 | 11p15.5 | Colon cancer cell line | Primary tumors |
| MYOD1 | 11p15.4 | AGING Colon. 100% colon, 30% | |
| | | breast, Also bladder, lung, liquid | |
| | | tumors. | |
| N33 | 8p22 | AGING Colon. 60-80% colon, | Oligo-saccharyl-transferase |
| | | prostate, brain. | |
| NEP (Neutral | 3q21-27 | Prostate cancer (~10%) | |
| Endopeptidase 24.1)/ | | | |
| CALLA | | | |
| NF-L (light-neurofilament- | 8p21 | Rat Glioma cell line | |
| encoding gene) | | | |
| NIS (sodium-iodide | 19p13.2-p12 | Thyroid cancer cell lines | Heterogeneous methylation in |
| symporter gene) | | | primary tumors |
| P14/ARF | 9p21 | Colon cancer cell lines | Less frequent than P16 |
| | | (infrequent) | methylation, but usually |
| | | | associated with P16 methylation. |
| P15 (CDKN2B) | 9p21 | 80% AML/ALL 2-20% GBM 0% | P15 is physically close to P16, |
| | | Colon/Lung/Breast | but simultaneous methylation of |
| | | | both genes is rare. |
| P16 (CDKN2A) | 9p21 | 20-30% Lung (NSCLC) 25-35% | Methylation is as frequent as |
| | | Colon 5-25% Lymphomas | deletions, and more frequent |
| | | (depending on stage) 0-5% | than mutations. |
| | | Bladder. Many others (esophagus, | |
| | | stomach etc.) | |
| P27KIP1 | 12p13 | Rodent pituitary cancer cell lines | No primary tumors reported |
| p57 KIP2 | 11p15.5 | Gastric cancer cell lines | |
| PAX6 | 11p13 | Colon cancer cell lines and 70% of | |
| | | primary tumors | |
| PgR (Progesterone | 11q22 | 10-20% Breast cancer | Effect on transcription |
| Receptor) | | | |
| RAR-Beta2 | 3p24 | Colon, Breast, Lung Cancer | |
| RASSF1 | 3p21.3 | Lung cancer | One promoter only |
| RB1 | 13q14 | 10-20% Retinoblastomas 0% | |
| (Retinoblastoma) | | Lung/Leukemia/Colon Some | |
| | | pituitary adenomas | |
| TERT | 5p15.33 | Heterogeneous methylation in | |
| | | many cell lines | |
| TESTIN | 7q31.2 | Hematopoietic malignancies | One promoter only |
| TGFBRI | 9q33-q34 | Gastric cancer cell lines and | |
| | | primary tumors (10%) | |
| THBS1 | 15q15 | 5-10% Colon Cancer 30-40% | Angiogenesis inhibitor, regulated |
| (Thrombospondin-1) | | GBM 20-30% AML 0% | by P53 and Rb in some systems. |
| | | Endometrial/Breast | |
| TIMP3 | 22q12.1-13.2 | Human brain (10-50%) and kidney | |
| | | (20%) cancers, Mouse model | |
| TLS3 | X | Leukemia cell lines | |
| (T-Plastin) | | | |
| Urokinase (uPA) | 10q24 | Breast cancer cell lines | |
| VHL | 3p25-25 | 10-20% Renal Cell cancers 0% | Same tumor selectivity as |
| (Von-Hippell Lindau) | | Common solid and liquid tumors | mutations |
| WT1 | 11p13 | 90% Breast cancers, 20-50% | Correlation with expression |
| | | colon, 5-10% Wilm's | |
| ZO2 | | Pancreatic cancer cell lines | |
| (Zona Occludens 2) | | | |

As will be noted from the above Table, the prior art has appreciated that methylation is important in the pathogenesis of cancer, however only diploid material has been used to date. Applicant proposes that methods of the prior art are susceptible to providing less than accurate information. For example, upon profiling a given promoter in a tumor sample, a moderate degree of methylation may be noted. In real terms the degree of methylation measured is an average of methylation for the maternally derived promoter sequence and the paternally derived promoter sequence, since the tumor is of course diploid. While this averaged result may be true in some cases (because both maternal and paternally derived promoters are methylated to the same extent), this will not be universally true. For example, the maternal promoter sequence could be completely unmethylated, and the paternal sequence may be heavily methylated. If the maternally-derived sequence is dominant over the paternal sequence in terms of promoter activity, then the importance of analyzing the maternal and paternal sequences separately becomes apparent.

It will be understood from the foregoing that the presence or absence of an epigenetic modification is analyzed with reference to the haploid state of the cell. Thus, the presence or absence of methylation at a given DNA site under consideration may determine where maternally derived epigenetic feature is substantially separated from the counterpart paternally derived epigenetic features. In this way, the influence of maternally derived epigenetic features can be ascertained in the absence of paternally derived epigenetic features, and vice versa. Applicant proposes that a more accurate methylation map is gained by removing the potentially inaccurate (or at least less than definitive) results provided where diploid material is analyzed.

The skilled artisan will appreciate that the present invention is distinguished from the natural process of genomic imprinting whereby the level of expression of some genes depends on whether or not they are inherited from the maternal or paternal genome. For example, insulin-like growth factor-2 (IGF2) is a gene whose expression is required for normal fetal development and growth. Expression of IGF2 occurs exclusively from the paternal copy of the gene. Imprinted genes are “marked” by their state of methylation. In the case of IGF2 an element in the paternal locus, called an insulator element, is methylated blocking its function. The function of the un-methylated insulator is to bind a protein that when bound blocks activation of IGF2 expression. When methylated, the protein cannot bind the insulator thus allowing a distant enhancer element to drive expression of the IGF2 gene. In the maternal genome, the insulator is not methylated, thus protein binds to it blocking the action of the distant enhancer element. By contrast, the present invention is concerned with providing a definitive haploid methylation assignment of maternal DNA, paternal DNA or both, by providing phase-specific information on methylation.

By “definitive”, it is meant that no estimate or inference or determination of likelihood or probability is involved in assigning a certain epigenetic modification pattern to a certain phenotype. Methods of analyzing epigenetic modifications described in the prior art are confounded by the presence of paternal modifications in combination with maternal modifications. Thus, while certain algorithms may be used to deduce or infer a likely assignment between a given methylation pattern and phenotype for example, where non-haploid material is analyzed these assignments will necessarily be flawed. It is proposed that the confusion often noted in assigning a methylation pattern to a phenotype results at least in part by the confounding influence of diploid material.

According to the present method, a DNA and/or associated protein is obtained from a biological sample of the subject. The biological sample may be any material that contains DNA and/or associated protein including but not limited to whole blood, serum, a blood cell, a skin cell, saliva, urine, hair, nails, tears, nails, and the like.

Where the step of analyzing includes the substantial isolation of paternally-derived DNA from maternally-derived, the skilled artisan may use any suitable method known to him or her. It should be understood that the means for achieving substantial isolation is not restrictive on the scope of the present invention, but in one form of the method the step of substantially isolating the paternally- or maternally-derived DNA and/or associated protein is by physical means. The advantage provided by physical means over non-physical means (such as selective probing of diploid material) is that problems associated with discerning maternally derived material from paternally derived material are avoided. For example, where selective probes are used, it could be that cross-hybridization is problematic leading to uncertain results. While hybridisation conditions can be varied to limit cross-hybridisation, this requires further experimentation to be performed.

In one form of the invention, the physical method to provide haploid DNA containing exclusively paternally-derived DNA or maternally-derived DNA is chromosome microdissection. The haploid DNA may be an entire maternal or paternal chromosome, a chromatid or a fragment of a chromatid. Conveniently, cuts in the chromosome may be made distal to the centromere to separate the p and q arms, each being a haploid DNA molecule. The skilled person is enabled to identify the physical region of interest in a chromosome and adopt an appropriate method to isolate haploid DNA from a diploid, tripolid, tetraploid, or any other sample having a higher level of ploidy.

The skilled person is familiar with platforms and tools used for micromanipulation. Although technically exacting, microdissection is routinely achievable. Equipment requirements consist of a microscope (either upright or inverted) fitted with a micromanipulator and a rotating stage, and a pipette puller (to produce microneedles). Vibration isolation for the microscope is recommended. Although a special clean room is not required, microdissected chromosome fragments contain only femptogram quantities of DNA, and contamination with extraneous DNA must be controlled.

In one form of the method, a non-contact method for isolating a haploid DNA molecule may be used. An example of this approach is the use of a laser microbeam. Laser microbeam microdissection may involve use of a pulsed ultraviolet laser of high beam quality interfaced with a microscope. Laser beam microdissection may be performed using, for example, a commercially available P.A.L.M.® Robot Microbeam (P.A.L.M. GmbH Bernried, Germany). The light laser is preferably of a wavelength that does not damage or destroy the genome segment, such as 337 nm which is remote from the absorption maximum of nucleic acids such as DNA.

Another useful system for laser microdissection of chromosomes is the Leica Laser Microdissection Microscope. The system uses a DMLA upright microscope including motorized nosepiece, motorized stage, the xyz-control element and all other advantages of the new DMLA microscope. The laser used is a UV laser of 337 nm wavelength. The movement during cutting is done by the optics, while the stage remains stationary. The region of interest can be marked on the monitor and is cut out by PC control. The sample falls down into PCR tubes without extra forces. The result of the cutting can be easily checked by an automated inspection mode.

Isolation of a haploid DNA molecule may be achieved by, for example, microdissection using laser catapulting of a chromosome segment using a PALM laser. In this case, the non-contact process involves laser ablation around the targeted chromosome element, followed by laser force catapulting of the defined element onto a tube cap, such as a microcentrifuge tube cap, for subsequent analysis of single arm DNA.

The resultant isolated haploid DNA molecule may be recovered using laser pressure catapulting. Laser pressure catapulting may be achieved by focussing a laser microbeam under, for example a haploid genome segment or segments of interest, and generating a force as a result of the high photon density that develops and causes the required haploid material to be catapulted from the non-required genome segment. The sample travels on the top of a photonic wave and is catapulted into a collection tube. Suitable collection tubes will be known to those of skill in the art and include tubes such as a common polymerase chain reaction (PCR) reaction tube or a microcentrifuge tube.

The paternally- or maternally-derived DNA may be substantially isolated by preparative flow cytometry using probes capable of discriminating between maternal and paternal DNA. Another method is by the use of radiation hybrids, where the development of diploid material involves human chromosomes as only one of each chromosome pair. Another strategy is the use of “conversion technology”, as developed by GMP Technologies Inc. GMP Conversion Technology® utilizes a process to separate paired chromosomes into single chromosomes. When separated, alleles may be analyzed individually using genetic probes that identify gene sequences. This technology is applicable to a gene, a chromosome, or to the entire human genome.

In another form of the method the contaminant genetic material is inactivated or ablated such that it no longer performs the function of contaminant genetic material. For example, where it is desired to isolate a maternally-derived DNA, the paternal contribution may be inactivated or ablated. This may be achieved by destroying a homologous chromosome using a carefully directed laser beam for example.

Another method for selectively analysing a paternally- or maternally-derived DNA molecule is to selectively amplify a haploid sequence using PCR such that the number of copies of haploid DNA is in vast excess over that of the contaminant DNA. The mixture of DNA molecules could then be partially digested with a nuclease such that substantially all contaminant DNA is digested, and a low level of haploid DNA remains.

The possibility also exists for selectively amplifying the haploid DNA by long PCR using primers incorporating a tag, and separating out the copies using the tag.

Once the paternally- or maternally-derived DNA is substantially isolated, analysis is undertaken to determine the presence or absence of an epigenetic modification.

Where the epigenetic modification is the methylation of DNA, methylation may be detected by analyzing the number 5 carbon of the cytosine pyrimidine ring for the presence or absence of a methyl group.

The method may be implemented using any suitable methodology, however typically the step of analyzing the one or more sites for the presence or absence of methylation comprises a method selected from the group consisting of DNA sequencing using bisulfite treatment, restriction landmark genomic scanning, methylation-sensitive arbitrarily primed PCR, Southern analysis using a methylation-sensitive restriction enzyme, methylation-specific PCR, restriction enzyme digestion of PCR products amplified from bisulfite-converted DNA, and combinations thereof.

Bisulfite sequencing involves reacting single-stranded DNA with sodium bisulfite, which selectively deaminates cytosine to uracil but does not react with methylcytosine. The modified DNA sequence produced in the bisulfite reaction is amplified by PCR, and then the amplified DNA is ligated into a plasmid vector for cloning and sequencing. When the DNA is sequenced, only the intact methylated cytosine residues are amplified as cytosine.

Bisulfite sequencing can be performed using DNA isolated from fewer than 100 cells, which is one of the major advantages of this tool, because tumor specimens are typically very small. Other benefits of bisulfite sequencing include its ability to analyze long stretches of the genome to determine very clear patterns of methylation in the DNA, and it yields a quantitative positive display of 5-methylcytosine residues.

MSP is a very rapid and sensitive technique for methylation screening. MSP is performed using sodium bisulfite to modify the DNA and convert unmethylated cytosines to uracil. Subsequent amplification is performed with primers specific for the methylated versus unmethylated DNA, and the analysis is performed with simple gel electrophoresis. MethyLight is the next generation of the MSP assay. The work up of the sample and the premise of the assays are identical. The MethyLight approach is an advance: While maintaining the exquisite sensitivity provided by standard MSP, the assay is made more quantitative, and less labor intensive through the incorporation of a real time “TaqMan” PCR format.

The most critical parameter affecting the specificity of methylation-specific PCR is determined by primer design. In practice, it is often preferred to deal with only one strand, most commonly the sense strand. In one form of the method, primers are designed to amplify a region that is 20-30 by in length, and should incorporate enough cytosines in the original sequence to assure that unmodified DNA will not serve as a template for the primers. In addition, the number and position of cytosines within the CpG dinucleotide determines the specificity of the primers for methylated or unmethylated templates. Typically, 1-3 CpG sites are included in each primer, and concentrated in the 3′ region of each primer. This provides optimal specificity and minimizes false positives due to mispriming. To facilitate simultaneous analysis of the U and M reactions of a given gene in the same thermocycler, we adjust the length of the primers to give nearly equal melting/annealing temperatures. This usually results in the U product being a few base pairs larger than the M product, which provides a convenient way to recognize each lane after electrophoresis.

Since methylation-specific PCR utilizes specific primer recognition to discriminate between methylated and unmethylated sites, it is preferred that stringent conditions are maintained for amplification. This means that annealing temperatures should be close to the maximum temperature which allows annealing and subsequent amplification. In practice, new primers are typically tested with an initial annealing temperature 5-8 degrees below the calculated melting temperature. Non-specificity can be remedied by slight increases in annealing temp, while lack or weak PCR products may be improved by a drop in temperature of 1-3 degrees Celsius. As with all PCR protocols, care should be taken to ensure that the template DNAs and reagents do not become contaminated with exogenous DNAs or PCR products.

MSP utilizes the sequence differences between methylated alleles and unmethylated alleles which occur after sodium bisulfite treatment. The frequency of CpG sites in CpG facilitate this sequence difference. Primers for a given locus are designed which distinguish methylated from unmethylated DNA in bisulfite-modified DNA. Since the distinction is part of the PCR amplification, extraordinary sensitivity, typically to the detection of 0.1% of alleles can be achieved, while maintaining specificity. Results are obtained immediately following PCR amplification and gel electrophoresis, without the need for further restriction or sequencing analysis. MSP also allows the analysis of very small samples, including paraffin-embedded and microdissected samples.

In one embodiment of the method the step of analyzing the one or more sites for the presence or absence of methylation analysis comprises: (a) reacting the haploid DNA sample with sodium bisulfite to convert unmethylated cytosine residues to uracil residues while leaving any 5-methylcytosine residues unchanged to create an exposed bisulfite-converted DNA sample having binding sites for primers specific for the bisulfite-converted DNA sample; (b) performing a PCR amplification procedure using top strand or bottom strand specific primers; (c) isolating the PCR amplification products; (d) performing a primer extension reaction using a Ms-SNuPE primer, dNTPs and Taq polymerase, wherein the Ms-SNuPE primer comprises from about a 15-mer to about a 22-mer length primer sequence that is complementary to the bisulfite-converted DNA sample and terminates immediately 5′ of the cytosine residue of the one or more CpG dinucleotide sequences to be assayed; and (f) determining the methylation state of the one or more CpG dinucleotide sequences by determining the identity of the first primer-extended base.

Methylation analysis may be facilitated by the use of high throughput methods to identify sites of potential methylation. Techniques available for screening include restriction landmark genome scanning (RLGS); gene expression arrays, which may used as a surrogate to see what genes are expressed after exposure to DNA methyltransferase inhibitors like azacitidine.

Highly parallel genome-wide assays are known in the art, with a number disclosed by Fan et al (Nat Rev Genet 2006, 7(8) 632-44; the contents of which is herein incorporated in its entirety). Many such methods are available to the skilled person as contract services, an example being the Golden Gate Methylation Solution” such as that provided by Illumina Inc (San Diego, Calif.). The Illumina system is capable of analyzing up to 1,536 CpG sites at single-site resolution over hundreds of genes across 96 samples. This system can therefore provide up to 147,456 quantitative DNA methylation measurements per assay.

It is further contemplated that a human CpG island library be obtained, and then arrayed. Database construction begins with crude chromatograms. After duplicates are removed, all 6,800 elements are subjected to Basic Local Alignment Search Tool (BLAST) analysis against the University of California, Santa Cruz, High Throughput Genomic and nr DNAsequence databases and were sequenced. In the CpG island database, each clone is assigned an identification number and its characteristics are listed, including such information as its chromosome location, whether it is a promoter or found in the 5′ flanking region, its GC content, and what restriction sites exist in the clone. The latter information is useful for determining the utility of restriction enzyme analysis as well as the actual sequence.

The CGI microarray technique is also contemplated to be useful in the context of the present methods. This technique permits simultaneous assessment of thousands of potential targets of DNA methylation on a single chip. It involves arraying of CpG island clones on glass slides, preparation of target sample amplicons, and hybridization of the amplicons onto the CGI microarrays. As an example, the technique may be performed using tumor derived genomic DNA samples that are obtained using a restriction enzyme (Mse1) able to cut immediately outside of the CpG island. Next, catch linkers bearing PCR primer sequences are added to the Mse1 fragments. The sample is split into a reference portion, which serves as a denominator to determine how well the genome amplification worked, and into a test portion that is digested with McrBC, a methylation sensitive restriction enzyme that only digests DNA if the region is methylated. The two portions are amplified by PCR, and then the DNA is direct labeled with Cy5 red (test) or Cy3 green (reference) fluorescent dyes. DNA fragments not digested by McrBC in the test sample produce Cy5-labeled PCR product while no labeled PCR product is produced if there were methylated fragments digested by McrBC. The labeled test and reference PCR products are mixed and spotted onto the glass slide. The hybridized slides are scanned and the acquired images analyzed to identify methylated signals.

As will be appreciated by the skilled person, the present invention will have many uses in the filed of biology, and particularly in medicine. As discussed supra, cancer is considered an epigenetic In fact, epigenetic changes, particularly DNA methylation, are susceptible to change and are excellent candidates to explain how certain environmental factors may increase the risk of cancer. The delicate organization of methylation and chromatin regulates the normal cellular homeostasis of gene expression patterns becomes unrecognizable in the cancer cell. The genome of the transformed cell may simultaneously undergo a global genomic hypomethylation and a dense hypermethylation of the CpG islands associated with gene regulatory regions. These dramatic changes may lead to chromosomal instability, activation of endogenous parasitic sequences, loss of imprinting, illegitimate expression, aneuploidy, and mutations, and may contribute to the transcriptional silencing of tumour suppressor genes. The hypermethylation-associated inactivation may affect many of the pathways in the cellular network, such as DNA repair (hMLH1, BRCA1, MGMT, em leader), the cell cycle (p16(INK4a), p14(ARF), p15(INK4b), and apoptosis (DAPK, APAF-1) The aberrant CpG island methylation can also be used as a biomarker of malignant cells and as a predictor of their behaviour.

In one form of the invention the haploid DNA is present in, or obtained, from a diploid cell. The method may use an autosomal chromosome of a somatic cell. The term “autosomal chromosome” means any chromosome within a normal somatic or germ cell except the sex chromosomes. For example, in humans chromosomes 1 to 22 are autosomal chromosomes. Applicant proposes that avoidance of naturally haploid material (such as that contained in sperm and ova) is advantageous because epigenetic modifications such as methylation are at least partially erased, and are often completely erased. This is because; the pattern of methylation is typically reset during meiosis. Thus, analysis of a sperm cell or ovum will not provide useful information allowing the definition of a phenotype for the subject.

The use of naturally haploid material such as sperm cells or ova is also to be avoided due to problems with obtaining these sex cells in the clinic. Obtaining ova is an invasive procedure for females of any age, and harvesting sperm cells from a pediatric male also requires medical intervention.

The avoidance of sex cells in epigenetic haplotyping has a further advantage when it is considered that during the process of meiosis recombination events may occur such that loci that were formerly linked in cis, become associated in trans. Thus, analysing an epigenetic haplotype of a gamete will give different (i.e. incorrect) haplotype information to that of haploid DNA obtained from a diploid cell.

In one form of the method, both paternally-derived and maternally-derived DNA and/or associated protein are analyzed for epigenetic modification. While information may be gained by investigating (for example) the methylation patterns on either maternal or paternal DNA, further information will be gained by analyzing DNA molecules from both sources for methylation.

In one embodiment of the invention, two or more sites on the same DNA molecule are analyzed for methylation. This form of the method is useful in determining whether the two methylated sites are naturally present in cis or in trans on the DNA molecule in the cell. This may be achieved by considering only the methylation sites on a single chromosome (either maternally or paternally-derived), for example to demonstrate that the two methylation sites are present in cis because they both appear on the same chromosome. It may also be necessary to analyze a maternal and paternal chromosome to demonstrate the two methylation sites are present in trans.

Alternatively, the method may be practiced where the DNA and/or associated protein is not physically separated into paternally- and maternally derived components. By using an in situ method it will be possible to selectively probe a paternal DNA or a maternal DNA such that it is unnecessary to physically separate the two molecules. In this way, diploid material may be used to provide information on the haploid state of the cell. Methods such as primed in situ labelling (PRINS) will be useful in this regard by selectively identifying maternal or paternal DNA thereby allowing the localisation of an epigenetic modification to a maternal or paternal chromosome. The method has been successfully used with primers specific for certain chromosomes, using both metaphase and interphase nuclei (Hindkjaer et al, Methods Mol Biol. 1994; 33:95-107).

It is also contemplated that PNA probes can be utilised for the in situ identification of chromosomes. PNA chemistry can be used to fabricate small oligomers. When dye-labeled, these oligomers make excellent probes and offer distinct advantages over conventional nucleic acids. PNA probes are typically very small (12 to 20 mers), and demonstrate both strong signals and very low background. PNA probes can be used in the context of fluorescence in situ hybridisation (PNA-FISH) as a powerful technology to explore chromosome structure on the metaphase and interphase chromosome. PNA-FISH is a method where small PNA oligomer probes are used to label chromosomes in a sequence specific manner, allowing identification of an underlying sequence in a particular chromosome. Methods for PNA-FISH are disclosed in Strauss 2002 (“PNA-FISH” in FISH Technology. Rautenstrauss/Liehr Eds. Springer Verlag. Heidelberg).

It will be understood that the methods described herein may find use in any area of biology where the effect of methylation and other protein modifications must be considered in connection with gene expression. These uses are not limited to animals (human or otherwise), and may also be applied to plants.

Phenotypic information obtained from the present methods includes the presence or absence of a disease, condition, or disorder, a predisposition to a disease, condition, or disorder, the ability or inability to respond to a therapeutic molecule, the ability or inability to mount an immune response against a foreign antigen or a self-antigen, the presence or absence of an allergy, a predisposition to an allergy.

The present invention will now be more fully described by reference to the following non-limiting examples.

### Example 1

### Methylation Specific PCR on Haploid DNA

Tissue Preparation

Metaphase spreads of peripheral blood cells obtained from a human subject are prepared by standard karyotyping methods onto PEN membrane slides. A standard Giemsa stain is prepared and used to identify chromosomes in the preparation.

Staining Procedure

| Step | Process | Time | |
|---|---|---|---|
| 1. | Phosphate buffer, pH6.8 | 1 | minute |
| 2. | 0.025% Trypsin in Phosphate Buffer, pH 6.8 | 2 | minutes |
| 3. | Phosphate Buffer, pH 6.8 | 1 | minute |
| 4. | Giemsa stain, working solution | 15 | minutes |
| 5. | Distilled water | 2-3 | dips |

Excess water is shaken off the slide, and the backside dried with a kimwipe and left to air dry.

Laser Microdissection

The PALM™ Microdissection system is used (P.A.L.M. Microlaser Technologies GmbH, Germany). Metaphase spreads (as prepared above) are used for laser capture using a P.A.L.M microscope under a 100× objective lens. Single metaphase chromosomes spatially separated from their sister chromosome, including single chromosomes are catapulted into the caps of 200 ul UltraFlux Flat Cap PCR tubes containing 6 μl of 0.1% (v/v) Triton-X-100 using standard P.A.L.M microscope protocols. The catapulted material was transferred to the bottom of the tube by centrifugation for methylation analysis. Both maternally-derived and paternally-derived DNA samples are obtained.

DNA Extraction

DNA from the microdissected chromosome fragment on the CapSure Macro LCM cap is extracted using PicoPure DNA Extraction Kit (Arcturus Inc, CA) following the kit protocol.

Detection of Methylation:

DNA is modified by sodium bisulfite treatment converting unmethylated, but not methylated, cytosines to uracil. Following removal of bisulfite and completion of the chemical conversion, this modified DNA is used as a template for PCR. Two PCR reactions are performed for each DNA sample, one specific for DNA originally methylated for the gene of interest, and one specific for DNA originally unmethylated. PCR products are separated on 6-8% non-denaturing polyacrylamide gels and the bands are visualized by staining with ethidium bromide. The presence of a band of the appropriate molecular weight indicates the presence of unmethylated, and/or methylated alleles, in the original sample.

To prepare the mixes 10× PCR buffer, NTP's and primers are thawed. The number of samples to be analyzed is determined, including a positive control for both the unmethylated and methylated reactions, and a water control. A master mix for each PCR reaction (methylated and unmethylated) is made. For each 50 μl reaction, the following amounts should be used:

| | | |
|---|---|---|
| 10x PCR buffer: | 5 | μL |
| 25 mM 4 NTP mix | 2.5 | μL |
| Sense primer (300 ng/μL) | 1 | μL |
| Antisense primer (300 ng/μL) | 1 | μL |
| Distilled, sterile water | 28.5 | μL |

38 μl of this PCR mix is dispensed into separate PCR tubes (0.5 mL tubes or strips) labeled for each sample. The components are well mixed prior to dispensing.

2 μl of bisulfite modified DNA template is added to each tube. An unmethylated and methylated reaction is included for each sample, as are positive controls, as well as a no DNA control.

One to two drops of mineral oil (˜25-50 μl) are added to each tube, before placing in thermocycler. The mineral oil completely covers the surface of the reaction mixture to prevent evaporation.

The PCR products are then amplified in the thermal cycler. The PCR is initiated with a five-minute denaturation at 95° C. Taq polymerase is added after initial denaturation: 1.25 units of Taq polymerase, diluted into 10 μl of sterile distilled water. This 10 μl is mixed into the 40 μl through the oil by gently by pipetting up and down. Amplification is continued for 35 cycles with the following parameters:

30 sec 95° C. (denaturation)

30 sec specific for primer (annealing)

30 sec 72° C. (elongation)

Final step: 4 min 72° C. (elongation)

Store at 40° C. until analysis.

The PCR products are analyzed by gel electrophoresis as follows.

A 6-8% non-denaturing polyacrylamide gel is prepared. 1× TBE provides better buffering capacity and sharper bands for resolving these products. The size of the products typically generated by MSP analysis is in the 80-200 by range, making acrylamide gels optimal for resolution of size. High-percentage horizontal agarose gels can be used as an alternative.

Reactions from each sample are run together to allow for direct comparison between unmethylated and methylated alleles. Positive and negative controls are included. Vertical gels are run at 10 V/cm for 1-2 hours. The gel is stained in ethidium bromide, and visualized under UV illumination. A comparison of the methylation status of maternally-derived DNA and paternally-derived DNA for a given DNA region is made.

Finally, it is to be understood that various other modifications and/or alterations may be made without departing from the spirit of the present invention as outlined herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1-15. (canceled)

16. A method for obtaining epigenetic information for a polyploid subject, the method including the steps of obtaining a biological sample from the subject, the sample containing:
(i) at least one paternally-derived DNA molecule and/or associated protein and/or,
(ii) at least one maternally-derived DNA molecule and/or associated protein, analyzing any one or more of the paternally- or maternally-derived DNA molecules or associated proteins for the presence or absence of modifications, wherein the step of analyzing determines whether any two modifications are present in cis on one chromosome, or in trans across two sister chromosomes.

17. A method according to claim 16, wherein the step of analyzing determines whether the modifications can be ascribed to the paternally-derived DNA and/or associated protein, or the maternally-derived DNA and/or associated protein.

18. A method according to claim 16, wherein the presence or absence of the modifications is capable of modulating expression of the DNA molecule in vivo.

19. A method according to claim 16, wherein the step of analyzing includes the substantial isolation of a paternally-derived DNA and/or associated protein from a maternally-derived DNA and/or associated protein using a physical method.

20. A method according to claim 19, wherein the physical method includes laser-mediated dissection of the paternally-derived DNA molecule and/or associated protein from the maternally-derived DNA molecule and/or associated protein.

21. A method according to claims 16, wherein the step of analyzing includes an in situ method capable of selectively analyzing paternally-derived DNA and/or associated protein as compared with maternally-derived DNA and/or associated protein.

22. A method according to claim 16, wherein the DNA molecule or associated protein is present in, or obtained, from a diploid cell.

23. A method according to claim 16, wherein where the step of analyzing is performed on DNA, the modification is methylation.

24. A method according to claim 23, wherein the analyzing includes determining methylation of a dinucleotide CpG.

25. A method according to claim 23, wherein the analyzing includes a method selected from the group consisting of DNA sequencing using bisulfite treatment, restriction landmark genomic scanning, methylation-sensitive arbitrarily primed PCR, Southern analysis using a methylation-sensitive restriction enzyme, methylation-specific PCR, restriction enzyme digestion of PCR products amplified from bisulfite-converted DNA, and combinations thereof.

26. A method according to claim 25, wherein where the analyzing includes DNA sequencing using bisulphite treatment, the analyzing includes: (a) reacting the DNA with sodium bisulfite to convert unmethylated cytosine residues to uracil residues while leaving any 5-methylcytosine residues unchanged to create an exposed bisulfite-converted DNA sample having binding sites for primers specific for the bisulfite-converted DNA sample; (b) performing a PCR amplification procedure using top strand or bottom strand specific primers; (c) isolating the PCR amplification products; (d) performing a primer extension reaction using a Ms-SNuPE primer, dNTPs and Taq polymerase, wherein the Ms-SNuPE primer comprises from about a 15-mer to about a 22-mer length primer sequence that is complementary to the bisulfite-converted DNA sample and terminates immediately 5′ of the cytosine residue of the one or more CpG dinucleotide sequences to be assayed; and (f) determining the methylation state of the one or more CpG dinucleotide sequences by determining the identity of the first primer-extended base.

27. A method according to claim 26, wherein the dNTPs are labeled, and determining the identity of the first primer-extended base is measured by incorporation of the labeled dNTPs.

28. A method according to claim 16, wherein where the analyzing is performed on protein, the protein is a histone and the modification is acetylation.

29. A method according to claim 16, wherein the epigenetic information is capable of providing phenotypic information for the subject.

30. A method according to claim 29, wherein the phenotypic information is selected from the group consisting of the presence or absence of a disease, condition, or disorder; a predisposition to a disease, condition, or disorder; the ability or inability to respond to a potentially therapeutic molecule; the ability or inability to mount an immune response against a foreign antigen or a self-antigen; the presence or absence of an allergy; a predisposition to an allergy.
